(19) United States
(12) Patent Application Publication
(10) Pub. No.: US 2019/0011457 A1
(43) Pub. Date: Jan. 10, 2019

(54) BIOMARKERS FOR DIAGNOSING KELOID SKIN OR KELOID SCAR, AND USE THEREOF

(71) Applicant: Tego Science Inc., Seoul (KR)

(72) Inventors: Saewha JEON, Seoul (KR); Yun Hee KIM, Seoul (KR); Jikhyon HAN, Seoul (KR); Hong Joo CHO, Goyang-si, Gyeonggi-do (KR); Eun Jung SHIN, Seocho-gu, Seoul (KR)

(21) Appl. No.: 16/070,121

(22) PCT Filed: Jan. 13, 2017
(86) PCT No.: PCT/KR2017/000472

§ 371 Date: Jul. 13, 2018

(30) Foreign Application Priority Data
Jan. 15, 2016 (KR) 10-2016-0005397

(51) Int. Cl. G01N 33/68

(52) U.S. Cl. CPC G01N 33/6881; G01N 2800/20

(57) ABSTRACT

The present invention relates to: biomarker proteins for diagnosing scars; a composition for diagnosing scars by using the same; a biochip for diagnosing scars; and a method for obtaining information on the scars. Proteins identified by the present invention can be applied as biomarkers for screening scars. A patient with keloid scars can be diagnosed by using the biomarker. In addition, the biomarker can be used for treating a patient with keloid scars.

### TECHNICAL FIELD

The present invention relates to biomarkers for diagnosing keloid skin or a keloid scar, and a composition for diagnosing keloid skin or a keloid scar. In addition, the present invention relates to a biochip for diagnosing keloid skin or a keloid scar and a method for obtaining information on the same. In addition, the present invention relates to a pharmaceutical composition for preventing or treating keloid skin or a keloid scar.

### BACKGROUND ART

If a certain level or more of damage to the skin occurs, scars cannot be avoided to varying degrees. Though the degree of scarring may vary depending on individual skin characteristics, the most important factor is that it is affected by the degree of an initial wound. However, often when there is a problem during normal occurrence of scars, scars excessively grow, resulting in a decrease of the quality of life and uncomfortable symptoms.

Keloid is a disorder in which abnormal fibrous tissue densely grows while in a wound healing process after skin damage, and has a property of growing around and over the size of an original wound or a region in which inflammation occurs.

It is considered that keloid occurs due to a disorder occurring in a function of properly regulating and inhibiting a wound healing process. Keloid occurs mainly in blacks or pigmented races and occurs after a person with a genetic predisposition (approximately 15% of the total population) has a skin injury.

Keloidal skin may cause cosmetic problems, and interfere with the movement of joints in critical areas such as the face or joints since it has a wide range of invasion.

Keloids (also called keloid scars) appear clinically as a skin color, low pigmentation or hard erythematous nodules. Unlike hypertrophic scars, keloids may infiltrate the peripheral normal skin beyond the original wound area.

The histopathological diagnosis of keloids (or keloid scars) is as follows. The epidermis is normal, and the dermis is proliferated, thick, abundant in blood vessels and increased in infiltration of inflammatory cells, compared with normal scar tissue. Collagen bundles of the normal dermis are relaxed and disorganized, whereas the keloidal dermis has thick and plentiful collagen bundles. The most characteristically histological finding of keloids is that collagen fibers consist of numerous fibrils and are densely arranged in a large and wide region. A thick, hyalinized collagen that is irregularly arranged in a spiral shape is called keloidal collagen.

In addition to the collagen, proteoglycans, which are one of the most important extracellular matrix components, are also over-deposited. Generally, the important findings for the histopathological diagnosis of a keloid are the presence of hyalinized collagen, the infiltration of a tongue-shaped collagen bundle that penetrates beneath the normal epidermis and papillary dermis, a horizontal cellular fibrous band seen in the upper dermis, and a prominent fascial band.

Hypertrophic scars refer to an overgrowth of collagen on an affected area that is larger than a normal size while wound healing, and they are deep-colored, protruding and make soft scars. The hypertrophic scar develops quickly after trauma, and improves over time. In addition, the hypertrophic scar is localized to the wound region, occurs frequently and is not related to skin color.

Until now, keloid skin or a keloid scar was diagnosed visually or histologically, and was not diagnosed using a biomarker.

Accordingly, the inventors continued a study of biomarkers for diagnosing scars such as keloids, and thus completed the present invention.

### PRIOR ART DOCUMENT

Korean Patent No. 1505294

### DISCLOSURE

### Technical Problem

An object of the present invention is to provide a composition for diagnosing keloid skin or a keloid scar.

Another object of the present invention is to provide a biochip for diagnosing keloid skin or a keloid scar, which includes the composition.

Still another object of the present invention is to provide a method for obtaining information on keloid skin or a keloid scar.

Yet another object of the present invention is to provide a pharmaceutical composition for preventing or treating keloid skin or a keloid scar.

### Technical Solution

To achieve the above objects, the present invention provides a composition for diagnosing keloid skin or a keloid scar, which includes any one or more peptides selected from the group consisting of peptides disclosed in the GenPept database at the National Center for Biotechnology Information (NCBI), shown in Tables 1 and 2, as an active ingredient:

TABLE 1

| | | GenPept | |
|---|---|---|---|
| | | Accession | |
| No. | Protein name | no. | Related link |
| 1 | 26S proteasome non- | NP_002808 | http://www.ncbi.nlm.nih.gov/protein/NP_002808 |
| | ATPase regulatory | | |
| | subunit 13 isoform 1 | | |
| 2 | 40S ribosomal protein | NP_001007 | http://www.ncbi.nlm.nih.gov/protein/NP_001007 |
| | S12 | | |
| 3 | Apo Form of Human | 3NXA_A | http://www.ncbi.nlm.nih.gov/protein/3NXA_A |
| | S100a16 | | |
| 4 | ASPRV1 protein | AAH31997 | http://www.ncbi.nlm.nih.gov/protein/AAH31997 |
| 5 | bifunctional purine | NP_004035.2 | http://www.ncbi.nlm.nih.gov/protein/NP_004035.2 |
| | biosynthesis protein | | |
| | PURH | | |
| 6 | Cellular retinoic acid | 2CBS_A | http://www.ncbi.nlm.nih.gov/protein/2CBS_A |
| | binding protein li in | | |
| | complex with synthetic | | |
| | retinoic acid | | |
| 7 | Aldehyde Reductase | 2ALR_A | http://www.ncbi.nlm.nih.gov/protein/2ALR_A |
| 8 | Human epidermal fatty | 4AZM_A | http://www.ncbi.nlm.nih.gov/protein/4AZM_A |
| | acid-binding protein | | |
| | (fabp5) in complex with | | |
| | the inhibitor Bms- | | |
| | 309413 | | |
| 9 | Prolyl oligopeptidase | 3DDU_A | http://www.ncbi.nlm.nih.gov/protein/3DDU_A |
| | with Gsk552 | | |
| 10 | Solution structure of | 2L50_A | http://www.ncbi.nlm.nih.gov/protein/2L50_A |
| | Apo S100a16 | | |
| 11 | Structure of [r563a] | 1SQM_A | http://www.ncbi.nlm.nih.gov/protein/1SQM_A |
| | Leukotriene A4 | | |
| | hydrolase | | |
| 12 | The high resolution | 1AXN_A | http://www.ncbi.nlm.nih.gov/protein/1AXN_A |
| | structure of annexin Iii | | |
| | shows differences with | | |
| | Annexin V | | |
| 13 | Three crystal structures | 1T2L_A | http://www.ncbi.nlm.nih.gov/protein/1T2L_A |
| | of human coactosin-like | | |
| | protein | | |
| 14 | Cystatin-B | NP_000091 | http://www.ncbi.nlm.nih.gov/protein/NP_000091 |
| 15 | Ezrin | AAH68458 | http://www.ncbi.nlm.nih.gov/protein/AAH68458 |
| 16 | Isopentenyl-diphosphate | Q13907 | http://www.ncbi.nlm.nih.gov/protein/Q13907 |
| | delta-isomerase 1; | | |
| | Short = IPPI1 | | |
| 17 | Keratin, type I | NP_000413 | http://www.ncbi.nlm.nih.gov/protein/NP_000413 |
| | cytoskeletal 17 | | |
| 18 | Macrophage-capping | XP_515584 | http://www.ncbi.nlm.nih.gov/protein/XP_515584 |
| | protein isoform 9 | | |
| 19 | Mitochondrial ATP | ABD77240 | http://www.ncbi.nlm.nih.gov/protein/ABD77240 |
| | synthase, H+ | | |
| | transporting F1 complex | | |
| | beta subunit | | |
| 20 | NCOR1 protein, partial | AAH58511 | http://www.ncbi.nlm.nih.gov/protein/AAH58511 |
| 21 | Phosphoglycerate | AAH62302 | http://www.ncbi.nlm.nih.gov/protein/AAH62302 |
| | mutase 1 (brain) | | |
| 22 | PREDICTED: tubulin | XP_002823231 | http://www.ncbi.nlm.nih.gov/protein/XP_002823231.1?report=genpept |
| | alpha-1B chain-like | | |
| | isoform 2 | | |
| 23 | Protein S100-A8 | XP_001138065 | http://www.ncbi.nlm.nih.gov/protein/XP_001138065.2?report=genpept |
| | isoform 4 pPan | | |
| | troglodytes] | | |
| 24 | R33729_1 | AAC27824 | http://www.ncbi.nlm.nih.gov/protein/AAC27824 |
| 25 | TALDO1 protein | AAH18847 | http://www.ncbi.nlm.nih.gov/protein/AAH18847 |
| 26 | Type I keratin 16 | AAB35421 | http://www.ncbi.nlm.nih.gov/protein/AAB35421 |
| 27 | All taxonomy (crystal | 3V03_A | http://www.ncbi.nlm.nih.gov/protein/3V03_A |
| | structure of bovine | | |
| | serum albumin) | | |
| 28 | Alpha-actinin-4 | NP_004915 | http://www.ncbi.nlm.nih.gov/protein/NP_004915 |
| 29 | Alpha-crystallin B chain | NP_001876 | http://www.ncbi.nlm.nih.gov/protein/NP_001876 |
| 30 | Crystal structure of | 3B97_A | http://www.ncbi.nlm.nih.gov/protein/3B97_A |
| | human enolase 1 | | |
| 31 | Human peroxiredoxin 5 | 1HD2_A | http://www.ncbi.nlm.nih.gov/protein/1HD2_A |
| 32 | Structural and | 1HVE_A | http://www.ncbi.nlm.nih.gov/protein/1HVE_A |
| | electrophysiological | | |
| | analysis of annexin V | | |
| | Mutants. mutagenesis of | | |
| | human annexin V | | |
| 33 | Structure of S100a4 in | 4ETO_A | http://www.ncbi.nlm.nih.gov/protein/4ETO_A |
| | complex with non- | | |
| | muscle myosin-Iia | | |
| | peptide | | |

TABLE 2

| | | GenPept | |
|---|---|---|---|
| No. | Protein name | Accession no. | Related link |
| 64 | Chain A, structure of | 4ETO_A | http://www.ncbi.nlm.nih.gov/protein/4ETO_A |
| | S100a4 in complex with | | |
| | non-muscle myosin-Iia | | |
| | peptide | | |
| 65 | Phosphoglycerate mutase | AAH62302 | http://www.ncbi.nlm.nih.gov/protein/AAH62302 |
| | 1 (brain) | | |
| 66 | Putative G-protein | BAB89334 | http://www.ncbi.nlm.nih.gov/protein/BAB89334 |
| | coupled receptor | | |
| 67 | Chain A, human quinone | 1QR2_A | http://www.ncbi.nlm.nih.gov/protein/1QR2_A |
| | reductase type 2 | | |
| 68 | T-plastin polypeptide | AAB02844 | http://www.ncbi.nlm.nih.gov/protein/AAB02844 |
| 69 | FLNA protein | AAH14654 | http://www.ncbi.nlm.nih.gov/protein/AAH14654 |
| 70 | Transgelin variant | BAD92792 | http://www.ncbi.nlm.nih.gov/protein/BAD92792 |
| 71 | hCG38213, isoform | EAW76181 | http://www.ncbi.nlm.nih.gov/protein/EAW76181 |
| | CRA_d | | |
| 72 | Leukotriene A4 | EAW97557 | http://www.ncbi.nlm.nih.gov/protein/EAW97557 |
| | hydrolase, isoform | | |
| | CRA_a | | |
| 73 | Prolyl 4-hydroxylase | NP_001017962 | http://www.ncbi.nlm.nih.gov/protein/NP_001017962 |
| | subunit alpha-1 isoform | | |
| | 2 precursor | | |
| 74 | Ubiquitin carboxyl- | NP_004172 | http://www.ncbi.nlm.nih.gov/protein/NP_004172 |
| | terminal hydrolase | | |
| | isozyme L1 | | |
| 75 | Rho GDP-dissociation | NP_004300 | http://www.ncbi.nlm.nih.gov/protein/NP_004300 |
| | inhibitor 1 isoform a | | |
| 76 | Cytokeratin-14 | P02533 | http://www.ncbi.nlm.nih.gov/protein/P02533 |
| 77 | Keratin 5 | AAH24292 | http://www.ncbi.nlm.nih.gov/protein/AAH24292 |
| 78 | Prohibitin | AAS88903 | http://www.ncbi.nlm.nih.gov/protein/AAS88903 |
| 79 | FLJ00410 protein | BAC03467 | http://www.ncbi.nlm.nih.gov/protein/BAC03467 |
| 80 | Serpin B5 | NP_002630 | http://www.ncbi.nlm.nih.gov/protein/NP_002630 |

The peptides shown in Tables 1 and 2 are registered in the GenPept database at NCBI, and the sequence of each peptide may be confirmed in a related link.

The peptide may be a peptide whose expression is increased or decreased two-fold or higher in skin cells in a scar region as compared with normal skin cells without a keloid scar. Specifically, any one or more peptides of the peptides 1 to 26 and the peptides 64 to 75 may be peptides whose expression is increased two-fold or higher in skin cells of a keloid scar region as compared with normal skin cells without a keloid scar, and any one or more peptides of the peptides 27 to 63 and the peptides 76 to 80 may be peptides whose expression is decreased two-fold or higher in skin cells in the keloid scar region, as compared with normal skin cells without a keloid scar.

The “peptide” used herein refers to a linear molecule formed by linking amino acid residues by a peptide bond. The term “peptide” used herein is widely used to mean the same as a “polypeptide” or “protein.”

The term “keloid” used herein refers to a disorder in which abnormal fibrous tissue densely grows while in a wound healing process after skin damage, and has a property of growing around and over the size of an original wound or inflammation-occurring region.

The term “keloidal skin” used herein refers to skin tissue in which the above-mentioned keloid occurs.

The term “keloid scar” used herein refers to a scar appearing as a keloid. Generally, the “scar” is the evidence of healing of damaged skin. A scar generated by excessively proliferating collagen of the dermis layer maintaining the skin tension when a deep layer of the dermis is damaged by surgery or trauma, and arising through the thinner skin even after a wound is healed is called a general scar, and such an abnormally-occurring process results in a keloid. Such a scar is called a keloid scar.

In addition, the present invention provides a biochip for diagnosing keloid skin or a keloid scar, which includes the composition.

In addition, the present invention provides a method for obtaining information on keloid skin or a keloid scar from a subject, which includes:

(a) measuring one or more biomarkers in a biological sample from a subject, wherein one or more biomarkers are selected from the compositions including a peptide, disclosed in Tables 1 and 2; and

(b) correlating measured value(s) with keloid skin or a keloid scar by comparing the measured value(s) with that/those of a normal person.

The term “subject” refers to a human, and preferably a patient or normal person who wants to obtain information on keloid skin or a keloid scar, but the present invention is not necessarily limited thereto.

The term “biological sample” refers to cells isolated from skin tissue of a subject, and specifically, a keratinocyte or fibroblast isolated from skin tissue of a subject, but the present invention is not limited thereto.

The “biomarker” refers to an index that can detect a change in the body using a protein, DNA, RNA or a metabolite, and biomarkers identified in the present invention may be a biomarker peptide for diagnosing keloid skin or a keloid scar.

The “measurement” may mean direct detection of the presence of a biomarker peptide or an immunogenic fragment thereof by two-dimensional electrophoresis, indirect confirmation of the presence of a biomarker peptide or an immunogenic fragment thereof by an antigen-antibody reaction, or quantification of an expression level of a biomarker peptide using a known method that can be used in protein quantification.

The antigen-antibody reaction may be performed using a known immunoassay method such as ELISA (coated tube), a magnetic particle method for quantifying an enzyme reaction occurring due to a competitive reaction of an antigen-tracer and a non-degradable contaminant by binding a magnetic particle to a tube, or a latex particle method using an antibody-binding latex particle.

The “measured value(s)” refer(s) to a result obtained by measurement in Step (a), and detection of the absence or presence of or measurement of an expression level of a biomarker peptide in a sample.

In Step (b), the measured value(s) of any one or more peptides selected from the group consisting of the peptides 1 to 26 and 64 to 75 shown in the table of claim 1 may be higher than that/those of a normal person, and correlated with a scar.

In Step (b), the measured value(s) of any one or more peptides selected from the group consisting of the peptides 27 to 63 and 76 to 80 shown in the table of claim 1 may be lower than that/those of a normal person and correlated with a scar.

The “being correlated with keloid skin or a keloid scar” means that the occurrence or probability of the occurrence of keloid skin or a keloid scar is expected.

In another aspect, the present invention provides a pharmaceutical composition for preventing or treating keloid skin or a keloid scar, which includes a peptide inhibitor for inhibiting the function of at least one selected from the group consisting of the peptides 1 to 26 and 64 to 75 shown in Tables 1 and 2 or the expression of a gene encoding the peptide, as an active ingredient.

In still another aspect, the present invention provides a pharmaceutical composition for preventing or treating keloid skin or a keloid scar, which includes a material that increases the expression of at least one selected from the group consisting of the peptides 27 to 63 and 76 to 80 shown in Tables 1 and 2 as an active ingredient.

One or two or more of the inhibitors or materials that increase expression may be included.

The inhibitor may include any material that inhibits at least one peptide selected from the group consisting of the peptides 1 to 26 and 64 to 75 shown in Tables 1 and 2. Specifically, the inhibitor may be an aptamer, a small compound, an antibody, or a functional fragment of the antibody which regulates the expression of any one selected from the group consisting of the peptides 1 to 26 and 64 to 75, or a viral vector, a non-viral vector, an antisense oligonucleotide, miRNA, siRNA or shRNA that regulates the expression of a gene encoding the peptide, but the present invention is not necessarily limited thereto.

The material that increases the expression of any one selected from the group consisting of the peptides 27 to 63 and 76 to 80 shown in Tables 1 and 2 may include any one of the materials that increase the expression of the peptide. Specifically, the material may include one selected from the peptides 27 to 63 and 76 to 80, a gene encoding the peptide, or a viral vector, non-viral vector, protein or small-molecule compound regulating the expression of the peptide, but the present invention is not limited thereto.

The term “peptide inhibitor” used herein has a meaning encompassing materials that decrease the expression or activation of at least one selected from the group consisting of the peptides 1 to 26 and 64 to 75 shown in Tables 1 and 2. More specifically, the peptide inhibitor may include all materials that decrease the expression or activation of the peptide by reducing the expression of the above-mentioned peptide at a transcription level or inhibiting the activation of the peptide by direct application to the above-mentioned peptide or indirect application to a ligand thereof. The material for inhibiting the peptide expression can be a compound, nucleic acid, peptide or virus which can inhibit the expression or activation of a peptide by targeting the peptide, or a vector including the nucleic acid regardless of its form. As an example of the material that inhibits the peptide expression, an oligonucleotide that inhibits mRNA expression of the peptide, an antibody that inhibits the activation of the peptide, or an antigen-binding fragment thereof is preferably used.

The term “antibody” used herein refers to a protein molecule including an immunoglobulin molecule having immunological reactivity with a specific antigen, and a protein molecule that serves as an antigen receptor specifically recognizing and reacting with an antigen when the specific antigen invades the body. One antibody material consists of two heavy chains and two light chains, and each of these heavy chains and light chains includes a variable domain and a constant domain. The variable domain includes three complementarity determining regions (CDRs) and four framework regions (FRs), and binding specificity of an antibody with respect to a specific antigen may be generated by forming a binding site between the antigen and antibody due to the CDRs.

The antibody that can be used in the present invention may be any antibody that inhibits the activity of the above-described peptide (at least one selected from the group consisting of the peptides 1 to 26 and 64 to 75 shown in Tables 1 and 2) without particular limitation, and may include, for example, a polyclonal antibody, a monoclonal antibody or an antibody fragment. In addition, antibodies which are manufactured by a genetic engineering method, for example, a chimeric antibody or a hybrid antibody, may also be used.

The functional fragments of the antibody may be Fab, F(ab′)2, Fab′, Fv, scFv and sdAb.

The term “oligonucleotide” used herein refers to a polymer that is formed by polymerization of several to tens of nucleotides using a phosphodiester bond. An example of an oligonucleotide that inhibits the expression of the above-mentioned peptide (at least one selected from the group consisting of the peptides 1 to 26 and 64 to 75 shown in Tables 1 and 2) is preferably an antisense oligonucleotide, an aptamer or small interfering RNA (siRNA), which is specific to the above-mentioned peptide, but the present invention is not limited thereto. As the oligonucleotide used in the present invention, siRNA is more preferably used.

The term “antisense oligonucleotide” used herein refers to DNA, RNA or a derivative thereof which contains a nucleic acid sequence complementary to the sequence of specific mRNA, and serves to inhibit translation of mRNA into a protein by binding to a complementary sequence in mRNA. In the present invention, an oligonucleotide which inhibits the expression of mRNA corresponding to the above-mentioned peptide may be DNA or RNA having a complementary sequence to mRNA of the above-mentioned peptide, or a derivative thereof, and may bind to mRNA of the peptide to interfere with translation, translocation into the cytoplasm or maturation of the peptide mRNA or to inhibit all biological function or activity of the peptide mRNA. The antisense oligonucleotide of the present invention may be suitably selected and used according to the type of peptide selected from the group consisting of the peptides 1 to 26 and 64 to 75 shown in Tables 1 and 2.

A length of the antisense oligonucleotide is not particularly limited, but is preferably 6 to 100 bases, more preferably 8 to 60 bases, and further more preferably 10 to 40 bases. The antisense oligonucleotide may be synthesized in vivo or synthesized in vitro and administered in vivo according to a conventional method used in the art. As a non-limiting example of the method for in vivo synthesis of antisense RNA, a method of transcribing antisense RNA using a vector in a direction opposite to the origin of a multi-cloning site (MCS) may be used. As a non-limiting example of the method for in vitro synthesis of antisense RNA, a method using RNA polymerase I may be used.

The antisense oligonucleotide which inhibits the expression of mRNA of the peptide of the present invention may be easily manufactured according to a known method by those of ordinary skill in the art with reference to the peptide base sequence of the present invention.

The term “aptamer” used herein refers to a single-stranded oligonucleotide, and an oligonucleotide molecule that has binding activity to a predetermined target molecule. The aptamer may have various three-dimensional structures according to a base sequence, and a high affinity to a specific material such as an antigen-antibody reaction. The aptamer may bind to a specific target molecule to inhibit the activity of a predetermined target molecule.

The aptamer of the present invention may be RNA, DNA, a modified oligonucleotide or a mixture thereof and may be formed in a linear or cyclic shape, but the present invention is not limited thereto. The aptamer of the present invention may be suitably selected according to the type of peptide of the present invention. The aptamer that inhibits the expression of mRNA of the peptide of the present invention may be easily manufactured according to a known method by those of ordinary skill in the art with reference to the base sequence of the peptide.

The term “small interfering RNA (siRNA)” used herein refers to a nucleic acid molecule that can mediate RNA interference or gene silencing, and a small double-stranded RNA fragment with a size of 20 to 25 nucleotides. The double strand of siRNA has a structure in which two nucleotides at the 3′-end overhang. Since siRNA can inhibit the expression of a target gene, the siRNA may be used as an effective gene knockdown method or gene therapy method. The siRNA that can be generated by cleaving double-stranded RNA with a dicer may specifically bind to mRNA having a complementary sequence and thus inhibit the expression of corresponding mRNA.

The siRNA of the present invention may be suitably selected according to the type of peptide used in the present invention (at least one selected from the group consisting of the peptides 1 to 26 and 64 to 75 shown in Tables 1 and 2). For example, in the case of aldehyde reductase among the peptides disclosed in the present invention, the siRNA may be siRNA having a sequence capable of specifically binding to mRNA of the aldehyde reductase. As long as the siRNA can inhibit the expression of aldehyde reductase mRNA, the sequence thereof is not particularly limited.

The siRNA of the peptide that can be used in the present invention may be designed or selected using a known database or program according to a target.

The siRNA of the peptide that can be used in the present invention may be easily manufactured according to a known method by those of ordinary skill in the art with reference to the base sequence of the peptide (at least one selected from the group consisting of the peptides 1 to 26 and 64 to 75 shown in Tables 1 and 2). A non-limiting example of the method for manufacturing siRNA may be a method for chemically synthesizing siRNA, a method for synthesizing siRNA using in vitro transcription, a method for manufacturing siRNA by cleaving long double-stranded RNA synthesized by in vitro transcription with a dicer, a method for expressing in vivo delivery of an shRNA expression plasmid or viral vector or an expression method through in vivo delivery of a polymerase chain reaction (PCR)-induced siRNA expression cassette.

The term “prevention” used herein refers to all actions that inhibit or delay the occurrence of keloid skin or a keloid scar by administering a composition including a material that inhibits the expression of the above-mentioned peptide of the present invention (at least one selected from the group consisting of the peptides 1 to 26 and 64 to 75 shown in Tables 1 and 2) as an active ingredient to a subject.

In addition, the term “prevention” used herein refers to all actions of inhibiting or delaying the occurrence of keloid skin or a keloid scar by administering a material that increases the expression of any one selected from the group consisting of the peptides 27 to 63 and 76 to 80 shown in Tables 1 and 2 to a subject.

The term “treatment” used herein refers to all actions involved in alleviating or beneficially changing symptoms of a keloid skin or keloid scar disorder by administration of the composition including a material that inhibits the occurrence of keloid skin or a keloid scar of the present invention as an active ingredient to a subject suspected of having keloid skin or a keloid scar.

A content of a peptide inhibitor or a material for increasing peptide expression, which is included in the pharmaceutical composition of the present invention may be, but is not particularly limited to, preferably 0.1 pmol/L to 1 mg/mL.

The pharmaceutical composition of the present invention may further include a pharmaceutically acceptable carrier, and may be formulated with the carrier to be provided as foods, medicines, feed additives and drinking water additives. The term “pharmaceutically acceptable carrier” used herein refers to a carrier or diluent that does not inhibit biological activity and characteristics of the administered compound, without stimulation of an organism.

A type of the carrier that can be used in the present invention is not particularly limited, and any pharmaceutically acceptable carrier that is conventionally used in the art can be used. A non-limiting example of the carrier may be saline, distilled water, Ringer's solution, buffered saline, an albumin injectable solution, a dextrose solution, a maltodextrin solution, glycerol or ethanol. The carrier may be used alone or in combination of two or more thereof.

In addition, when necessary, the pharmaceutical composition may be used by adding other conventional additives such as an antioxidant, a buffer solution and/or a bacteriostatic agent, and may be formulated in the form of an injectable form such as an aqueous solution, a suspension, an emulsion, or a pill, a tablet, a capsule, a granule or a tablet by further adding a diluent, a dispersant, a surfactant, a binder and/or a lubricant.

A method for administering the pharmaceutical composition of the present invention may be performed according to a method conventionally used in the art without particular limitation. A non-limiting example of the administration method may be oral administration or parenteral administration of the composition.

The pharmaceutical composition may be manufactured in various forms depending on a desired administration method. A non-limiting example of a dosage form for oral administration may be a troche, a lozenge, a tablet, an aqueous suspension, an oil suspension, prepared powder, a granule, an emulsion, a hard capsule, a soft capsule, a syrup or an elixir.

To formulate the composition of the present invention in the form for oral administration such as a tablet or capsule, the composition may include a binder such as lactose, saccharose, sorbitol, mannitol, starch, amylopectin, cellulose or gelatin; an excipient such as dicalcium phosphate; a disintegrating agent such as corn starch or sweet potato starch; or a lubricant such as magnesium stearate, calcium stearate, sodium stearyl fumarate or polyethylene glycol wax. Further, in the case of a capsule, in addition to the above-mentioned materials, a liquid carrier such as fatty oil may be further contained.

The composition of the present invention may be administered by a parenteral method, for example, intravenous administration, intraperitoneal administration, intramuscular administration, subcutaneous administration or local administration, and the composition may be administered by applying or spraying on a disease area, but the present invention is not limited thereto.

As a dosage form for parenteral administration, for example, the composition may be formulated as an injectable form for subcutaneous injection, intravenous injection or intramuscular injection; a suppository; or a spray type such as an aerosol, which can be used for inhalation with a respirator, but the present invention is not limited thereto. To be formulated as the injectable form, the composition of the present invention is mixed with a stabilizer or a buffering agent in water and thus prepared as a solution or suspension, and may be prepared for ampoule- or vial-unit administration. To formulate the composition as a spray type such as an aerosol, an additive such as a propellant may be additionally mixed to disperse a water-dispersed concentrate or wet powder.

The term “subject” used herein means all animals including a human which has or is likely to have keloid skin or a keloid scar.

The preventing or treating method of the present invention may be administration of the composition according to the present invention at a pharmaceutically acceptable amount, specifically, to a subject which has or is likely to have keloid skin or a keloid scar. The daily dose of the composition may be suitably determined by those of ordinary skill in the art within the scope of correct medical judgment.

Specifically, a pharmaceutically effective amount of the composition for a specific animal may be determined, taking into account, the type and degree of a response to be achieved, the age, body weight, general health condition or diet of a corresponding subject, the administration time, route and secretion rate of the composition of the present invention, and the duration of treatment, and may vary depending on various factors and similar factors well known in the medicine field.

The administration route and method of the composition are not particularly limited, and may conform to any administration route and method if the composition can reach a desired corresponding region. Specifically, the composition may be administered via various oral or parenteral routes, and as a non-limiting example of the administration route, the composition may be administered orally, intrarectally, locally, intravenously, intraperitoneally, intramuscularly, intraarterially, transdermally, intranasally or by inhalation.

A suitable application, spray, or dosage of the pharmaceutical composition of the present invention may vary according to a preparation method, administration method, administration time and/or administration route of the composition, the age, body weight or sex of an animal for administration, the severity of a disease, food intake, or an excretion rate, and the dosage effective in desired treatment may be easily determined and prescribed by those of ordinary skill in the art.

In another aspect, the present invention provides a quasi-drug for preventing or improving keloid skin or a keloid scar, which includes a peptide inhibitor for inhibiting the function of at least one selected from the group consisting of the peptides 1 to 26 and 64 to 75 shown in Tables 1 and 2, or the expression of a gene encoding the peptide.

In another aspect, the present invention provides a quasi-drug for preventing or improving keloid skin or a keloid scar, which includes a material for increasing the expression of at least one selected from the group consisting of the peptides 27 to 63 and 76 to 80 shown in Tables 1 and 2.

The inhibitor and the material for increasing expression are the same as described above.

The term “improvement” used herein refers to all types of actions that at least reduce parameters related to a condition to be treated, for example, a degree of a symptom.

The term “quasi-drug” used herein refers to a product that is less active than drugs, among the products used for the purpose of diagnosing, treating, improving, alleviating, curing or preventing a disease of a human or animal. For example, according to the Pharmaceutical Affairs Law, quasi-drugs are textile and rubber products used for the treatment or prevention of a human or animal disease, non-apparatus or non-machinery products that slightly or indirectly act on a human body, or a sterilizer/pesticide for preventing an infectious disease, excluding products that are used for drugs. The type or formulation of the quasi-drug composition of the present invention is not particularly limited, and may be preferably an antibacterial cleanser, a shower foam, a mouthwash, a wet tissue, a detergent (soap), a handwash, a humidifier sterilizer, a mask, an ointment or a filter paper.

According to still another aspect of the present invention, a nutraceutical food composition for preventing or improving keloid skin or a keloid scar is provided.

n the nutraceutical food composition of the present invention is used as a food supplement, the composition may be added alone or in combination with another food or food ingredient, and may be properly used according to a conventional method.

The type of food includes, but is not particularly limited to, all types of foods in a usual meaning. Non-limiting examples of foods to which the above-mentioned material can be added may include meat, sausage, bread, chocolate, candy, snacks, confectionery, pizza, ramen, noodles, gum, dairy products including ice cream, various kinds of soups, beverages, tea, drinks, alcoholic beverages and multi vitamins.

n the functional and nutraceutical food composition of the present invention is a beverage composition, the composition may contain various flavoring agents or natural carbohydrates like a common beverage as an additional ingredient. Non-limiting examples of the natural carbohydrates may include monosaccharides such as glucose and fructose; disaccharides such as maltose and sucrose; natural sweetening agents such as dextrin and cyclodextrin; and synthetic sweetening agents such as saccharin and aspartame. A ratio of the additional ingredient added may be suitably determined by the choice of one of ordinary skill in the art.

In addition, the functional and nutraceutical food composition of the present invention may contain various nutrients, vitamins, electrolytes, flavoring agents, coloring agents, pectic acid and salts thereof, alginic acid and salts thereof, organic acids, protective colloid thickening agents, pH adjusting agents, stabilizers, stabilizers, glycerin, alcohol, or a carbonating agent used in a carbonated beverage. Moreover, the functional and nutraceutical food composition of the present invention may contain fruit flesh for preparing natural fruit juice, fruit-based beverages or vegetable-based beverages. Such an ingredient may be used alone or in combination of two or more thereof. The proportion of such an additive may also be suitably selected by one of ordinary skill in the art.

Unless defined otherwise, all technical and scientific terms used herein have the same meaning as commonly understood by those of ordinary skill in the art to which the present invention belongs. Generally, the nomenclature used herein is well known and commonly used in the art.

### Advantageous Effects

Proteins identified by the present invention can be applied as biomarkers for screening keloid skin or a keloid scar. Using the biomarker, patients with keloid skin or a keloid scar can be diagnosed. In addition, the biomarker can be used to treat a patient with keloid skin or a keloid scar.

### DESCRIPTION OF DRAWINGS

FIG. 1 is a 2D electrophoresis image for proteins extracted from keratinocytes isolated from normal skin tissue.

FIG. 2 is a 2D electrophoresis image for proteins extracted from keratinocytes isolated from skin tissue in a keloid scar region. FIG. 3 is a 2D electrophoresis image for proteins extracted from fibroblasts isolated from normal skin tissue.

FIG. 4 is a 2D electrophoresis image for proteins extracted from fibroblasts isolated from skin tissue in a keloid scar region.

### MODES OF THE INVENTION

Hereinafter, the present invention will be described in further detail with reference to examples. These examples are merely provided to illustrate the present invention, and it will be apparent to those of ordinary skill in the art that it should not be construed that the scope of the present invention is limited by the following examples.

### EXAMPLE 1

### Culture of Keratinocytes and Fibroblasts

1-1: Culture of Keratinocytes

Keratinocytes isolated from normal skin tissue and skin tissue in scar regions of patients with keloid scars were cultured on a feeder in a DMEM/F12 medium containing 10% fetal bovine serum (FBS) and 10 ng/ml EGF. When the cultured cells were grown to 70 to 80%, a feeder was removed and only the keratinocytes were isolated.

1-2: Culture of Fibroblasts

Fibroblasts isolated from normal skin tissue and skin tissue in scar regions of patients with keloid scars were cultured in an F12 medium containing 10% FBS. When the cultured cells were grown to 70 to 80%, only the fibroblasts were isolated.

### EXAMPLE 2

### Protein Extraction

A 10× volume of a sample solution consisting of 7M urea, 2M thiourea, 4%(w/v) 3-[(3-cholamidopropyl)dimethyammonio]-1-propanesulfonate (CHAPS), 1%(w/v) dithiothreitol (DTT), 2%(v/v) pharmalyte and 1 mM benzamidine was prepared. The keratinocytes or fibrocytes isolated in Example 1 were mixed with the prepared sample solution, and then the cells were disrupted using a homogenizer. In addition, for protein extraction, the cells were vortexed for 1 hour, centrifuged at 15° C. and 15,000 rpm for 1 hour, and then the supernatant was used as a sample for 2D electrophoresis.

### EXAMPLE 3

### Protein Expression Analysis using 2D Electrophoresis

For primary isoelectric focusing (IEF), IPG strips were reswelled with a reswelling solution consisting of 7M urea, 2M thiourea, 2% 3-[(3-cholamidopropyl)dimethyammonio]-1-propanesulfonate (CHAPS), 1% DTT and 1% pharmalyte at room temperature for approximately 12 to 16 hours. 200 μg of the sample per strip was used, and subjected to IEF at 20° C. For IEF, the voltage was increased from 150V to 3,500V within 3 hours, and the voltage was maintained at 3,500V for 26 hours, and then the focusing was completed after 96 kVh.

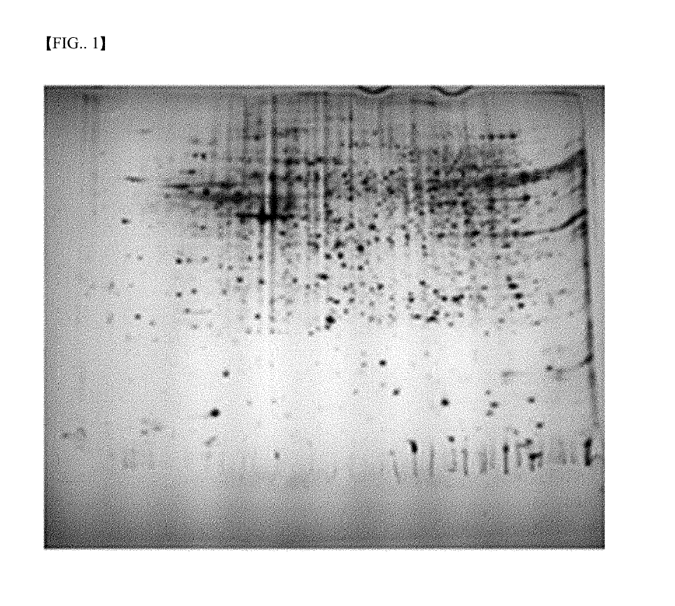

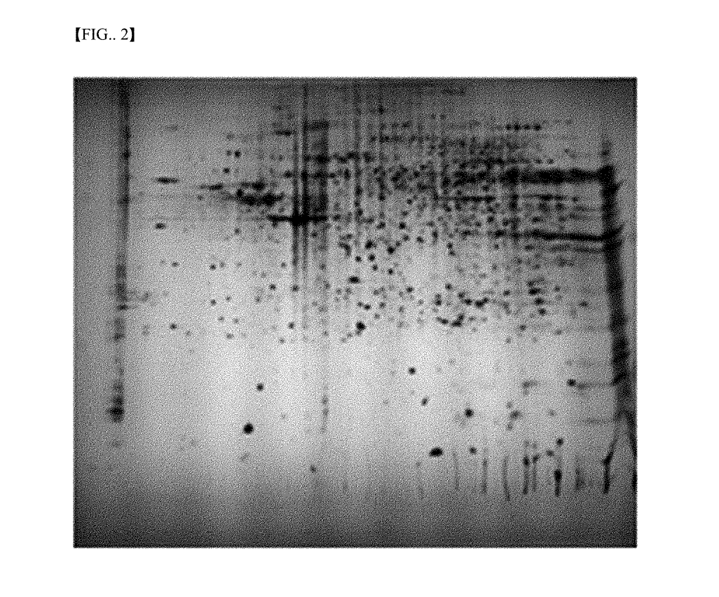

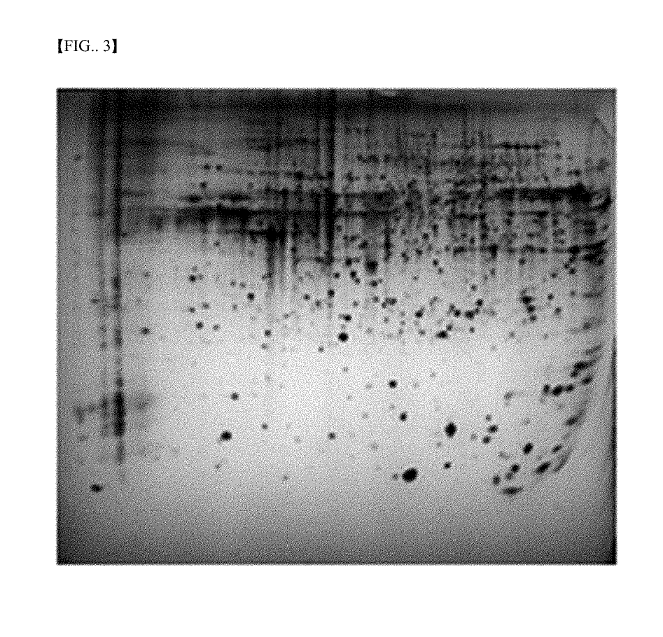

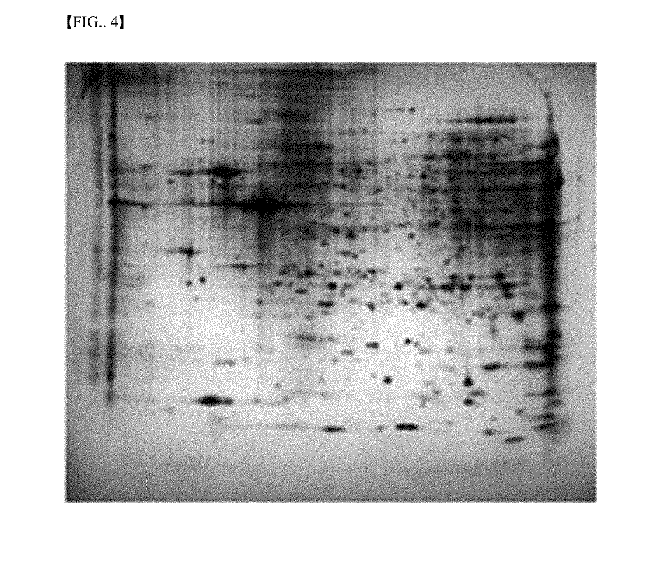

Before SDS-PAGE was performed secondarily, IPG strips were incubated in a 1% DTT-containing equilibration buffer (50 mM Tris-Cl, pH 6.8, 6M urea, 2% SDS, and 30% glycerol) for 10 minutes, and then further incubated in a 2.5% iodoacetamide-containing equilibration buffer for 10 minutes. Equilibrated strips were arranged on SDS-PAGE gels (20×24 cm, 10 to 16%), and run at 1.7 kVh and 20° C. using a Hoefer DALT 2D system. After 2D electrophoresis was completed, proteins on the 2D gels were visualized by silver staining, and a glutaraldehyde treatment step was omitted for protein identification by mass spectrometry. The silver-stained 2D gel was scanned, and stored in the form of a file with an extension, TIFF. FIG. 1 shows the 2DE image of normal keratinocytes, FIG. 2 shows the 2DE image of keloidal keratinocytes, FIG. 3 shows the 2DE image of normal fibroblasts, and FIG. 4 shows the 2DE image of keloidal fibroblasts.

A quantitative analysis for checking changes in protein spot expression from the scanned images was performed using PDQuest software. By comparing spots of the normal cell population and spots of the keloidal cell population, spots 2-fold or more increased or decreased compared to the normal cells were selected, and the result of performing protein identification for the selected spots are shown in the following Tables 3 (the proteins extracted from the keratinocytes) and 4 (the proteins extracted from the fibroblasts). Nos. 1 to 26 and 64 to 75 represent the proteins 2-fold or more increased compared to the normal cells, and Nos. 27 to 63 and 76 to 80 represent the proteins 2-fold or more decreased compared to the normal cells.

Protein peptide mass fingerprinting (PMF) identification using MALDI-TOF was performed through a known conventional method (Biomol Ther (Seoul), May 30, 2013; 21(3): 190-195). As a mass spectrometer, Microflex LRF 20 (Bruker Daltonics) was used. Protein fragments dropped on a target plate were vaporized by N2 laser irradiation at 337 nm and accelerated by a 20 Kv injection pulse. A mass spectrum for each protein spot was obtained by a cumulative peak of 300 laser shots. For analysis of the mass spectrum, an ion peak m/z (842.510, 2211.1046) of a peptide produced by the autolysis of trypsin was used as a standard peak. For protein identification from the analyzed mass spectrum, a MASCOT search engine (http://www.matrixscience.com) was used.

TABLE 3

| | | | Comparison |
|---|---|---|---|
| | | GenPept | with normal |
| No. | Protein name | Accession no. | (fold) |
| 1 | 26S proteasome non-ATPase regulatory subunit 13 | NP_002808 | 2.0 |
| | isoform 1 | | |
| 2 | 40S ribosomal protein S12 | NP_001007 | 2.3 |
| 3 | Apo form of human S100a16 | 3NXA_A | 2.6 |
| 4 | ASPRV1 protein | AAH31997 | 674.1 |
| 5 | Bifunctional purine biosynthesis protein PURH | NP_004035.2 | 4.0 |
| 6 | Cellular retinoic acid binding protein li in complex with | 2CBS_A | 2140.8 |
| | synthetic retinoic acid | | |
| 7 | Aldehyde reductase | 2ALR_A | 16.9 |
| 8 | Human epidermal fatty acid-binding protein (fabp5) in | 4AZM_A | 2.2 |
| | complex with the inhibitor Bms-309413 | | |
| 9 | Prolyl oligopeptidase with Gsk552 | 3DDU_A | 3.2 |
| 10 | Solution structure of Apo S100a16 | 2L50_A | 3562.0 |
| 11 | Structure of [r563a] leukotriene A4 hydrolase | 1SQM_A | 3.4 |
| 12 | The high resolution structure of annexin Iii shows | 1AXN_A | 1935.1 |
| | differences with annexin V | | |
| 13 | Three crystal stmctures of human coactosin-like protein | 1T2L_A | 4.0 |
| 14 | Cystatin-B | NP_000091 | 2.6 |
| 15 | Ezrin | AAH68458 | 5.4 |
| 16 | Isopentenyl-diphosphate delta-isomerase 1; Short = IPPI1 | Q13907 | 3.6 |
| 17 | Keratin, type I cytoskeletal 17 | NP_000413 | 12.2 |
| 18 | Macrophage-capping protein isoform 9 | XP_515584 | 4.8 |
| 19 | Mitochondrial ATP synthase, H+ transporting F1 | ABD77240 | 2.8 |
| | complex beta subunit | | |
| 20 | NCOR1 protein, partial | AAH58511 | 2.0 |
| 21 | Phosphoglycerate mutase 1 (brain) | AAH62302 | 2.4 |
| 22 | PREDICTED: tubulin alpha-1B chain-like isoform 2 | XP_002823231 | 821.6 |
| 23 | Protein S100-A8 isoform 4 [Pan troglodytes] | XP_001138065 | 3473.3 |
| 24 | R33729_1 | AAC27824 | 2.4 |
| 25 | TALDO1 protein | AAH18847 | 915.0 |
| 26 | Type I keratin 16 | AAB35421 | 195.4 |
| 27 | All taxonomy (Crystal Structure of Bovine Serum | 3V03_A | −5.76 |
| | Albumin) | | |
| 28 | Alpha-actinin-4 | NP_004915 | −4.70 |
| 29 | Alpha-crystallin B chain | NP_001876 | −5.43 |
| 30 | Crystal structure of human enolase 1 | 3B97_A | −2.00 |
| 31 | Human peroxiredoxin 5 | 1HD2_A | −12.14 |
| 32 | Structural and electrophysiological analysis of annexin V | 1HVE_A | −6.35 |
| | mutants. mutagenesis of human annexin V | | |
| 33 | Structure of S100a4 in complex with non-muscle | 4ETO_A | −13.12 |
| | myosin-Iia peptide | | |
| 34 | X-ray crystal structure of human galectin-1 | 1GZW_B | −4.15 |
| 35 | Structure of the human class I histocompatibility antigen, | 1HLA_M | −2.18 |
| | Hla-A2 | | |
| 36 | Chip-Ubc13-Uev1a complex | 2C2V_B | −4.38 |
| 37 | Chorionic somatomammotropin hormone-like 1 isoform 3 | NP_001309, | −2.04 |
| 38 | Dynactin 3 (p22) | CAI13144 | −3.72 |
| 39 | Eukaryotic translation initiation factor 2B, subunit 2 | AAH00494 | −7.66 |
| | beta, 39 kDa | | |
| 40 | Gelsolin isoform b | NP_937895 | −2.19 |
| 41 | Glutaredoxin-3 | NP_006532 | −4.21 |
| 42 | Glutathione S-transferase P | NP_000843 | −3.18 |
| 43 | Heat shock cognate 71 kDa protein isoform 1 | NP_006588 | −2.41 |
| 44 | HMOX2 | CAG33041 | −689.62 |
| 45 | Human Glutathione Transferase Omega 1, Delta 155 | 3LFL_A | −2.94 |
| 46 | Keratin 1, keratin, type I cytoskeletal 9 | AAG41947 | −950.95 |
| 47 | LMNB1 protein | AAH78178 | −540.70 |
| 48 | Moesin ferm domain bound to Ebp50 C-terminal peptide | 1SGH_A | −3.83 |
| 49 | Mrp14 complexed with chaps | 1IRJ_A | −4.07 |
| 50 | MYO5C protein | AAH64841 | −3.40 |
| 51 | Nicotinamide N-methyltransferase | NP_006160 | −5.79 |
| 52 | Nuclear transfactor 2 | NP_005787 | −9.35 |
| 53 | nucleoside diphosphate kinase A isoform a | NP_937818 | −19.07 |
| 54 | O-methyltransferase | 3BWM_A | −3.44 |
| 55 | Platelet-activating factor acetylhydrolase IB subunit | NP_002564 | −2.40 |
| | gamma | | |
| 56 | Protein S100-A11 | NP_005611 | −3.19 |
| 57 | Pyruvate dehydrogenase (lipoamide) beta | EAW65372 | −5.94 |
| 58 | Serine/threonine-protein phosphatase 2A catalytic | NP_058736 | −1331.25 |
| | subunit beta isoform | | |
| 59 | Tubulin alpha-1B chain isoform 2 | XP_002823231 | −7.94 |
| 60 | Tumor necrosis factor type 1 receptor associated protein | A55877 | −3.11 |
| | TRAP-1-human | | |
| 61 | Ubiquitin carboxyl-terminal hydrolase 14 isoform a | NP_005142 | −4.52 |
| 62 | Vacuolar protein sorting-associated protein 29 | NP_057310 | −2.52 |
| 63 | X-ray repair cross-complementing protein 5 | NP_066964 | −7.91 |

When the expression of Proteins 1 to 26 shown in Table 3 is increased, the sample can be diagnosed as the skin of a patient with keloid skin or a keloid scar, and when the expression of Proteins 27 to 63 is decreased, the sample can be diagnosed as the skin of a patient with keloid skin or a keloid scar.

TABLE 4

| | | | Comparison |
|---|---|---|---|
| | | GenPept | with normal |
| No. | Protein name | Accession no. | (fold) |
| 64 | Chain A, structure of S100a4 in complex with non- | 4ETO_A | 6575.08 |
| | muscle myosin-Iia peptide | | |
| 65 | Phosphoglycerate mutase 1 (brain) | AAH62302 | 16.35 |
| 66 | Putative G-protein coupled receptor | BAB89334 | 7.92 |
| 67 | Chain A, human quinone reductase type 2 | 1QR2_A | 4.63 |
| 68 | T-plastin polypeptide | AAB02844 | 10.81 |
| 69 | FLNA protein | AAH14654 | 5.17 |
| 70 | Transgelin variant | BAD92792 | 26.98 |
| 71 | hCG38213, isoform CRA_d | EAW76181 | 3.19 |
| 72 | Leukotriene A4 hydrolase, isoform CRA_a | EAW97557 | 6.94 |
| 73 | Prolyl 4-hydroxylase subunit alpha-1 isoform 2 | NP_001017962 | 4.54 |
| | precursor | | |
| 74 | Ubiquitin carboxyl-terminal hydrolase isozyme L1 | NP_004172 | 19.98 |
| 75 | Rho GDP-dissociation inhibitor 1 isoform a | NP_004300 | 2.75 |
| 76 | Cytokeratin-14 | P02533 | −2051.74 |
| 77 | Keratin 5 | AAH24292 | −1291.44 |
| 78 | Prohibitin | AAS88903 | −2.21 |
| 79 | FLJ00410 protein | BAC03467 | −2.32 |
| 80 | Serpin B5 | NP_002630 | −7150.72 |

When the expression of Proteins 64 to 75 shown in Table 4 is increased, the sample can be diagnosed as the skin of a patient with keloid skin or a keloid scar, and when the expression of Proteins 76 to 80 is decreased, the sample can be diagnosed as the skin of a patient with keloid skin or a keloid scar.

1. A composition for diagnosing keloid skin or a keloid scar, comprising:
any one or more peptides selected from the group consisting of the peptides disclosed in the GenPept database shown in the following table and thereof.

| | | GenPept |
|---|---|---|
| No | Protein name | Accession no. |
| 1 | 26S proteasome non-ATPase regulatory subunit 13 | NP_002808 |
| | isoform 1 | |
| 2 | 40S ribosomal protein S12 | NP_001007 |
| 3 | Apo Form Of Human S100a16 | 3NXA_A |
| 4 | ASPRV1 protein | AAH31997 |
| 5 | bifunctional purine biosynthesis protein PURH | NP_004035.2 |
| 6 | Cellular Retinoic Acid Binding Protein II In Complex | 2CBS_A |
| | With A Synthetic Retinotic Acid | |
| 7 | Aldehyde Reductase | 2ALR_A |
| 8 | Human Epidermal Fatty Acid-binding Protein (fabp5) In | 4AZM_A |
| | Complex With The Inhibitor Bms-309413 | |
| 9 | Prolyl Oligopeptidase With Gsk552 | 3DDU_A |
| 10 | Solution Structure Of Apo S100a16 | 2L50_A |
| 11 | Structure Of [r563a] Leukotriene A4 Hydrolase | ISQM_A |
| 12 | The High Resolution Structure Of Annexin III Shows | 1AXN_A |
| | Differences With Annexin V | |
| 13 | Three Crystal Structures Of Human Coactosin-Like | 1T2L_A |
| | Protein | |
| 14 | cystatin-B | NP_000091 |
| 15 | Ezrin | AAH68458 |
| 16 | Isopentenyl-diphosphate Delta-Isomerase 1; Short = IPPI1 | Q13907 |
| 17 | keratin, type I cytoskeletal 17 | NP_000413 |
| 18 | macrophage-capping protein isoform 9 | XP_515584 |
| 19 | mitochondrial ATP synthase, H+ transporting F1 | ABD77240 |
| | complex beta subunit | |
| 20 | NCOR1 protein, partial | AAH58511 |
| 21 | Phosphoglycerate mutase 1 (brain) | AAH62302 |
| 22 | PREDICTED: tublin alpha-1B chain-like isoform 2 | XP_002823231 |
| 23 | protein S100-A8 isoform 4 [Pan troglodytes] | XP_001138065 |
| 24 | R33729_1 | AAC27824 |
| 25 | TALDO1 protein | AAH18847 |
| 26 | type 1 keratin 16 | AAB35421 |
| 27 | all taxonomy (Crystal Structure Of Bovine Serum | 3V03_A |
| | Albumin) | |
| 28 | alpha-actinin-4 | NP_004915 |
| 29 | alpha-crystallin B chain | NP_001876 |
| 30 | Crystal Structure Of Human Enolase 1 | 3B97_A |
| 31 | Human Peroxiredoxin 5 | 1HD2_A |
| 32 | Structural And Electrophysiological Analysis Of Annexin | 1HVE_A |
| | V Mutants, Mutagenesis Of Human Annexin V | |
| 33 | Structure Of S100a4 In Complex With Non-Muscle | 4ETO_A |
| | Myosin-IIa Peptide | |
| 34 | X-Ray Crystal Structure Of Human Galectin-1 | 1GZW_B |
| 35 | Structure Of The Human Class 1 Histocompatibility | 1HLA_M |
| | Antigen, Hla-A2 | |
| 36 | Chip-Ubc13-Uevla Complex | 2C2V_B |
| 37 | chorionic somatomammotropin hormone-like 1 isoform 3 | NP_001309 |
| 38 | dynactin 3 (p22) | CAI13144 |
| 39 | Eukaryotic translation initiation factor 2B, subunit 2 | AAH00494 |
| | beta, 39 kDa | |
| 40 | gelsolin isoform b | NP_937895 |
| 41 | glutaredoxin-3 | NP_006532 |
| 42 | glutathione S-tranferase P | NP_000843 |
| 43 | heat shock cognate 71 kDa protein isoform 1 | NP_006588 |
| 44 | HMOX2 | CAG33041 |
| 45 | Human Glutathione Transferase Omega 1, Delta 155 | 3LFL_A |
| 46 | keratin 1, keratin, type I cytoskeletal 9 | AAG41947 |
| 47 | LMNB1 protein | AAH78178 |
| 48 | Moesin Ferm Domain Bound To Ebp50 C-Terminal | 1SGH_A |
| | Peptide | |
| 49 | Mrp14 Complexed With Chaps | 1IRJ_A |
| 50 | MYO5C protein | AAH64841 |
| 51 | nicotinamide N-methyltransferase | NP_006160 |
| 52 | Nucear transfactor 2 | NP_005787 |
| 53 | nucleoside diphosphate kinase A isoform a | NP_937818 |
| 54 | O-Methyltransferase | 3BWM_A |
| 55 | platelet-activating factor acetylhydrolase IB subunit | NP_002564 |
| | gamma | |
| 56 | protein S100-A11 | NP_005611 |
| 57 | pyruvate dehydrogenase (lipoamide) beta | EAW65372 |
| 58 | serine/threonine-protein phosphatase 2A catalytic | NP_058736, |
| | subunit beta isoform | |
| 59 | tubulin alpha-1B chain isoform 2 | XP_002823231 |
| 60 | tumor necrosis factor type 1 receptor associated | A55877 |
| | protein TRAP-1 - human | |
| 61 | ubiquitin carboxyl-terminal hydrolase 14 isoform a | NP_005142 |
| 62 | vacuolar protein sorting-associated protein 29 | NP_057310 |
| 63 | X-ray repair cross-complementing protein 5 | NP_066964 |
| 64 | Chain A, Structure Of S100a4 In Complex With | 4ETO_A |
| | Non-Muscle Myosin-IIa Peptide | |
| 65 | Phosphoglycerate mutase 1 (brain) | AAH62302 |
| 66 | putative G-protein coupled receptor | BAB89334 |
| 67 | Chain A, Human Quinone Reductase Type 2 | 1QR2_A |
| 68 | T-plastin polypeptide | AAB02844 |
| 69 | FLNA protein | AAH14654 |
| 70 | transgelin variant | BAD92792 |
| 71 | hCG38213, isoform CRA_d | EAW76181 |
| 72 | leukotriene A4 hydrolase, isoform CRA_a | EAW97557 |
| 73 | prolyl 4-hydroxylase subunit alpha-1 Isoform 2 | NP_001017962 |
| | precursor | |
| 74 | ubiquitin carboxyl-terminal hydrolase isozyme L1 | NP_004172 |
| 75 | rho GDP-dissociation inhibitor 1 isoform a | NP_004300 |
| 76 | Cytokeratin-14 | P02533 |
| 77 | Keratin 5 | AAH24292 |
| 78 | prohibitin | AAS88903 |
| 79 | FLJ00410 protein | BAC03467 |
| 80 | serpin B5 | NP_002630 |

2. A biochip for diagnosing keloid skin or a keloid scar, comprising: the composition of claim 1.

3. A method for obtaining information on keloid skin or a keloid scar from a subject, comprising:
(a) measuring one or more biomarkers in a biological sample obtained from a subject, wherein the one or more biomarkers are selected from the composition of claim 1; and
(b) correlating the measured value(s) with keloid skin or a keloid scar as compared with a normal person.

4. A method for obtaining information on keloid skin or a keloid scar from a subject, comprising:
(a) measuring one or more biomarkers in a biological sample obtained from a subject, wherein the one or more biomarkers are selected from the composition of claim 1; and
(b) correlating the measured value(s) with keloid skin or a keloid scar as compared with a normal person,
wherein, in Step (b), when measured values of any one or more peptides selected from the group consisting of the peptides 1 to 26 and 64 to 75 shown in the table of claim 1 are higher than those of a normal person, the values are correlated with keloid skin or a keloid scar.

5. A method for obtaining information on keloid skin or a keloid scar from a subject, comprising:
(a) measuring one or more biomarkers in a biological sample obtained from a subject, wherein the one or more biomarkers are selected from the composition of claim 1, and
(b) correlating the measured value(s) with keloid skin or a keloid scar as compared with a normal person,
wherein, in Step (b), when measured values of any one or more peptides selected from the group consisting of the peptides 27 to 63 and 76 to 80 shown in the table of claim 1 are lower than those of a normal person, the values are correlated with keloid skin or a keloid scar.

6. A pharmaceutical composition for preventing or treating keloid skin or a keloid scar, comprising:
a peptide inhibitor that inhibits the function of any one selected from the group consisting of the peptides 1 to 26 and 64 to 75 shown in the table below or the expression of a gene encoding the peptide, as an active ingredient, or
a material that increases the expression of any one selected from the group consisting of the peptides 27 to 63 and 76 to 80 shown in the table below, as an active ingredient.

| | | GenPept |
|---|---|---|
| No | Protein name | Accession no. |
| 1 | 26S proteasome non-ATPase regulatory subunit 13 | NP_002808 |
| | isoform 1 | |
| 2 | 40S ribosomal protein S12 | NP_001007 |
| 3 | Apo Form Of Human S100a16 | 3NXA_A |
| 4 | ASPRV1 protein | AAH31997 |
| 5 | bifunctional purine biosynthesis protein PURH | NP_004035.2 |
| 6 | Cellular Retinoic Acid Binding Protein II In Complex | 2CBS_A |
| | With A Synthetic Retinoic Acid | |
| 7 | Aldehyde Reductase | 2ALR_A |
| 8 | Human Epidermal Fatty Acid-binding Protein (fabp5) In | 4AZM_A |
| | Complex With The Inhibitor Bms-309413 | |
| 9 | Prolyl Oligopeptidase With Gsk552 | 3DDU_A |
| 10 | Solution Structure Of Apo S100a16 | 2L50_A |
| 11 | Structure Of [r563a] Leukotriene A4 Hydrolase | 1SQM_A |
| 12 | The High Resolution Structure Of Annexin III Shows | 1AXN_A |
| | Differences With Annexin V | |
| 13 | Three Crystal Structures Of Human Coactosin-Like | 1T2L_A |
| | Protein | |
| 14 | cystatin-B | NP_000091 |
| 15 | Ezrin | AAH68458 |
| 16 | Isopentenyl-diphosphate Delta-isomerase 1; Short = IPPI1 | Q13907 |
| 17 | keratin, type I cytoskeletal 17 | NP_000413 |
| 18 | macrophage-capping protein isoform 9 | XP_515584 |
| 19 | mitochondrial ATP synthase, H+ transporting F1 | ABD77240 |
| | complex beta subunit | |
| 20 | NCOR1 protein, partial | AAH58511 |
| 21 | Phosphoglycerate mutase 1 (brain) | AAH62302 |
| 22 | PREDICTED: tublin alpha-1B chain-like isoform 2 | XP_002823231 |
| 23 | protein S100-A8 isoform 4 [Pan troglodytes] | XP_001138065 |
| 24 | R33729_1 | AAC27824 |
| 25 | TALDO1 protein | AAH18847 |
| 26 | type I keratin 16 | AAB35421 |
| 27 | all taxonomy (Crystal Structure Of Bovine Serum | 3V03_A |
| | Albumin) | |
| 28 | alpha-actinin-4 | NP_004915 |
| 29 | alpha-crystallin B chain | NP_001876 |
| 30 | Crystal Structure Of Human Enolase 1 | 3B97_A |
| 31 | Human Peroxiredoxin 5 | 1HD2_A |
| 32 | Structural And Electrophysiological Analysis Of Annexin | 1HVE_A |
| | V Mutants, Mutagenesis Of Human Annexin V | |
| 33 | Structure Of S100a4 In Complex With Non-Muscle | 4ETO_A |
| | Myosin-IIa Peptide | |
| 34 | X-Ray Crystal Structure Of Human Galectin-1 | 1GZW_B |
| 35 | Structure Of The Human Class 1 Histocompatibility | 1HLA_M |
| | Antigen, Hla-A2 | |
| 36 | Chip-Ubc13-Uevla Complex | 2C2V_B |
| 37 | chorionic somatomammotropin hormone-like 1 isoform 3 | NP_001309 |
| 38 | dynactin 3 (p22) | CAI13144 |
| 39 | Eukaryotic translation initiation factor 2B, subunit 2 | AAH00494 |
| | beta, 39 kDa | |
| 40 | gelsolin isoform b | NP_937895 |
| 41 | glutaredoxin-3 | NP_006532 |
| 42 | glutathione S-tranferase P | NP_000843 |
| 43 | heat shock cognate 71 kDa protein isoform 1 | NP_006588 |
| 44 | HMOX2 | CAG33041 |
| 45 | Human Glutathione Transferase Omega 1, Delta 155 | 3LFL_A |
| 46 | keratin 1, keratin, type I cytoskeletal 9 | AAG41947 |
| 47 | LMNB1 protein | AAH78178 |
| 48 | Moesin Ferm Domain Bound To Ebp50 C-Terminal | ISGH_A |
| | Peptide | |
| 49 | Mrp14 Complexed With Chaps | 1IRJ_A |
| 50 | MYO5C protein | AAH64841 |
| 51 | nicotinamide N-methyltransferase | NP_006160 |
| 52 | Nucear transfactor 2 | NP_005787 |
| 53 | nucleoside diphosphate kinase A isoform a | NP_937818 |
| 54 | O-Methyltransferase | 3BWM_A |
| 55 | platelet-activating factor acetylhydrolase IB subunit | NP_002564 |
| | gamma | |
| 56 | protein S100-A11 | NP_005611 |
| 57 | pyruvate dehydrogenase (lipoamide) beta | EAW65372 |
| 58 | serine/threonine-protein phosphatase 2A catalytic | NP_058736, |
| | subunit beta isoform | |
| 59 | tubulin alpha-1B chain isoform 2 | XP_002823231 |
| 60 | tumor necrosis factor type 1 receptor associated | A55877 |
| | protein TRAP-1 - human | |
| 61 | ubiquitin carboxyl-terminal hydrolase 14 isoform a | NP_005142 |
| 62 | vacuolar protein sorting-associated protein 29 | NP_057310 |
| 63 | X-ray repair cross-complementing protein 5 | NP_066964 |
| 64 | Chain A, Structure Of S100a4 In Complex With | 4ETO_A |
| | Non-Muscle Myosin-IIa Peptide | |
| 65 | Phosphoglycerate mutase 1 (brain) | AAH62302 |
| 66 | putative G-protein coupled receptor | BAB89334 |
| 67 | Chain A, Human Quinone Reductase Type 2 | 1QR2_A |
| 68 | T-plastin polypeptide | AAB02844 |
| 69 | FLNA protein | AAH14654 |
| 70 | transgelin variant | BAD92792 |
| 71 | hCG38213, isoform CRA_d | EAW76181 |
| 72 | leukotriene A4 hydrolase, isoform CRA_a | EAW97557 |
| 73 | prolyl 4-hydroxylase subunit alpha-1 isoform 2 | NP_001017962 |
| | precursor | |
| 74 | ubiquitin carboxyl-terminal hydrolase isozyme L1 | NP_004172 |
| 75 | rho GDP-dissociation inhibitor 1 isoform a | NP_004300 |
| 76 | Cytokeratin-14 | P02533 |
| 77 | Keratin 5 | AAH24292 |
| 78 | prohibitin | AAS88903 |
| 79 | FLJ00410 protein | BAC03467 |
| 80 | serpin B5 | NP_002630 |

7. The pharmaceutical composition of claim 6, wherein the inhibitor is an aptamer, a small compound, an antibody or a functional fragment of the antibody, which regulates the expression of any one selected from the peptides 1 to 26 and 64 to 75, or a viral vector, a non viral vector, an antisense oligonucleotide, miRNA, siRNA or shRNA that regulates the expression of a gene encoding the peptide.

8. The pharmaceutical composition of claim 6, wherein the material that increases the expression of any one selected from the group consisting of the peptides 27 to 63 and 76 to 80 shown in the tables is a peptide selected from the group consisting of the peptides 27 to 63 and 76 to 80, a gene encoding the peptide, or a viral vector, non-viral vector, protein or small-molecule compound regulating the expression of the peptide.

9. A quasi-drug composition for preventing or improving keloid skin or a keloid scar, comprising:
a peptide inhibitor that inhibits the function of any one selected from the group consisting of the peptides 1 to 26 and 64 to 75 shown in the table below or the expression of a gene encoding the peptide, as an active ingredient, or
a material that increases the expression of any one selected from the group consisting of the peptides 27 to 63 and 76 to 80 shown in the table below, as an active ingredient.

| | | GenPept |
|---|---|---|
| No | Protein name | Accession no. |
| 1 | 26S proteasome non-ATPase regulatory subunit 13 | NP_002808 |
| | Isoform 1 | |
| 2 | 40S ribosomal protein S12 | NP_001007 |
| 3 | Apo Form Of Human S100a16 | 3NXA_A |
| 4 | ASPRV1 protein | AAH31997 |
| 5 | bifunctional purine biosynthesis protein PURH | NP_004035.2 |
| 6 | Cellular Retinoic Acid Binding Protein II In Complex | 2CBS_A |
| | With A Synthetic Retinoic Acid | |
| 7 | Aldehyde Reductase | 2ALR_A |
| 8 | Human Epidermal Fatty Acid-binding Protein (fabp5) In | 4AZM_A |
| | Complex With The Inhibitor Bms-309413 | |
| 9 | Prolyl Oligopeptidase With Gsk552 | 3DDU_A |
| 10 | Solution Structure Of Apo S100a16 | 2L50_A |
| 11 | Structure Of [r563a] Leukotriene A4 Hydrolase | 1SQM_A |
| 12 | The High Resolution Structure Of Annexin III Shows | 1AXN_A |
| | Differences With Annexin V | |
| 13 | Three Crystal Structures Of Human Coactosin-Like | 1T2L_A |
| | Protein | |
| 14 | cystatin-B | NP_000091 |
| 15 | Ezrin | AAH68458 |
| 16 | Isopentenyl-diphosphate Delta-Isomerase 1; Short = IPPI1 | Q13907 |
| 17 | keratin, type I cytoskeletal 17 | NP_000413 |
| 18 | macrophage-capping protein isoform 9 | XP_515584 |
| 19 | mitochondrial ATP synthase, H+ transporting F1 | ABD77240 |
| | complex beta subunit | |
| 20 | NCOR1 protein, partial | AAH58511 |
| 21 | Phosphoglycerate mutase 1 (brain) | AAH62302 |
| 22 | PREDICTED: tubulin alpha-1B chain-like isoform 2 | XP_002823231 |
| 23 | protein S100-A8 isoform 4 [Pan troglodytes] | XP_001138065 |
| 24 | R33729_1 | AAC27824 |
| 25 | TALDO1 protein | AAH18847 |
| 26 | type I keratin 16 | AAB35421 |
| 27 | all taxonomy (Crystal Structure Of Bovine Serum | 3V03_A |
| | Albumin) | |
| 28 | alpha-actinin-4 | NP_004915 |
| 29 | alpha-crystallin B chain | NP_001876 |
| 30 | Crystal Structure Of Human Enolase 1 | 3B97_A |
| 31 | Human Peroxiredoxin 5 | 1HD2_A |
| 32 | Structural And Electrophysiological Analysis Of Annexin | 1HVE_A |
| | V Mutants, Mutagenesis Of Human Annexin V | |
| 33 | Structural Of S100a4 In Complex With Non-Muscle | 4ETO_A |
| | Myosin-IIa Peptide | |
| 34 | X-Ray Crystal Structure Of Human Galectin-1 | 1GZW_B |
| 35 | Structure Of The Human Class I Histocompatibility | 1HLA_M |
| | Antigen, Hla-A2 | |
| 36 | Chip-Ubc13-Uev1a Complex | 2C2V_B |
| 37 | chorionic somatomammotropin hormone-like 1 isoform 3 | NP_001309 |
| 38 | dynactin 3 (p22) | CAI13144 |
| 39 | Eukaryotic translation initiation factor 2B, subunit 2 | AAH00494 |
| | beta, 39 kDa | |
| 40 | gelsolin isoform b | NP_937895 |
| 41 | glutaredoxin-3 | NP_006532 |
| 42 | glutathione S-transferase P | NP_000843 |
| 43 | heat shock cognate 71 KDa protein isoform 1 | NP_006588 |
| 44 | HMOX2 | CAG33041 |
| 45 | Human Glutathione Transferase Omega 1, Delta 155 | 3LFL_A |
| 46 | keratin 1, keratin, type I cytoskeletal 9 | AAG41947 |
| 47 | LMNB1 protein | AAH78178 |
| 48 | Moesin Ferm Domain Bound To Ebp50 C-Terminal | 1SGH_A |
| | Peptide | |
| 49 | Mrp14 Complexed With Chaps | 1IRJ_A |
| 50 | MYO5C protein | AAH64841 |
| 51 | nicotinamide N-methyltransferase | NP_006160 |
| 52 | Nucear transfactor 2 | NP_005787 |
| 53 | nucleoside diphosphate kinase A isoform a | NP_937818 |
| 54 | O-Methyltransferase | 3BWM_A |
| 55 | platelet-activating factor acetylhydrolase IB subunit | NP_002564 |
| | gamma | |
| 56 | protein S100-A11 | NP_005611 |
| 57 | pyruvate dehydrogenase (lipoamide) beta | EAW65372 |
| 58 | serine/threonine-protein phosphatase 2A catalytic | NP_058736, |
| | subunit beta isoform | |
| 59 | tubulin alpha-1B chain isoform 2 | XP_002823231 |
| 60 | tumor necrosis factor type 1 receptor associated | A55877 |
| | protein TRAP-1 - human | |
| 61 | ubiquitin carboxyl-terminal hydrolase 14 isoform a | NP_005142 |
| 62 | vacuolar protein sorting-assodated protein 29 | NP_057310 |
| 63 | X-ray repair cross-complementing protein 5 | NP_066964 |
| 64 | Chain A, Structure Of S100a4 In Comptex With | 4ETO_A |
| | Non-Muscle Myosin-IIa Peptide | |
| 65 | Phosphoglycerate mutase 1 (brain) | AAH62302 |
| 66 | putative G-protein coupled receptor | BAB89334 |
| 67 | Chain A, Human Quinone Reductase Type 2 | 1QR2_A |
| 68 | T-plastin polypeptide | AAB02844 |
| 68 | FLNA protein | AAH14654 |
| 70 | transgelin variant | BAD92792 |
| 71 | hCG38213, isoform CRA_d | EAW76181 |
| 72 | leukotriene A4 hydrolase, isoform CRA_a | EAW97557 |
| 73 | prolyl 4-hydroxylase subunit alpha-1 isoform 2 | NP_001017962 |
| | precursor | |
| 74 | ubiquitin carboxyl-terminal hydrolase isozyme L1 | NP_004172 |
| 75 | rho GDP-dissociation inhibitor 1 isoform a | NP_004300 |
| 76 | Cytokeratin-14 | P02533 |
| 77 | Keratin 5 | AAH24292 |
| 78 | prohibitin | AAS88903 |
| 79 | FLJ00410 protein | BAC03467 |
| 80 | serpin B5 | NP_002630 |
